(19) United States
(12) Patent Application Publication
(10) Pub. No.: US 2002/0170108 A1
(43) Pub. Date: Nov. 21, 2002

(54) SWIMMING GOGGLES

(76) Inventor: Herman Chiang, Chung-Ho City (TW)

Correspondence Address: Keith Kline, PRO-TECHTOR INTERNATIONAL SERVICES, 20775 Norada Court, Saratoga, CA, 95070-3018, US

(21) Appl. No.: 09/863,235

(22) Filed: May 21, 2001

(51) Int. Cl. A61F 9/02

(52) U.S. Cl. 2/442; 2/443

(57) ABSTRACT

A pair of swimming goggles for swimming pool purposes comprising: a left lens frame, a right lens frame, two lens units and a strap mean, each of lens frames has an inner periphery that defines a lens retaining space for receiving a lens unit and the periphery of each lens frame have at least one connection base. A strap mean for passing through the connection base of each of lens frames and tightening the swimming goggles to a wearer's head, such the swimming goggles can effectively guard against interference each other during the adjusting and can respectively conform with a wearer's eyehole contour, so as to provide more comfortable fitting and prevent the seepage of water when the swimming goggles are in use.

### FIELD OF THE INVENTION

The present invention relates to a swimming goggles, more particularly to a pair of swimming goggles which have two lens frames that can effectively guard against interference each other during the adjusting and can respectively conform with a wearer's eyehole contour, so as to provide more comfortable fitting and prevent the seepage of water when the swimming goggles are in use.

### BACKGROUND OF THE INVENTION

Conventionally, a swimming goggles has an adjustment element for adjusting a span between a left lens frame and a right lens frame. Nevertheless, this is so because the span between two eyes as well as the eyehole's contour may be different from person to person, thereby the left lens frame and the right lens frame of all types of swimming goggles can not respectively match wearer's eyehole contour and result the each lens frame unable to prevent the seepage of water when swimming goggles are in use. Referring to FIG. 1, a pair of swimming goggles 8 comprise a bridge 80, a right lens frame 81 and a left lens frame 82, the bridge 80 have two ends which respectively are connected to a connection base 83 of the right lens frame 81 and the left lens frame 82, which makes the right lens frame 81 and the left lens frame 82 influence each other when adjusting a span of the bridge. Moreover, the bridge 80 is limited the span which adjust between the right lens frame 81 and the left lens frame 82 in which the right lens frame 81 and the left lens frame 82 synchronously are moved with far away or closely each other, thereby adjusting the right lens frame 81 (or the left lens frame 82) to be in conformity with the periphery of right eyehole (or left eyehole) will move the left lens frame 82 (or the right lens frame 81) during the swimming goggles are worn on the wearer's eyeholes. In other words, the right lens frame 81 and the left lens frame 82 never respectively match wearer's eyehole contour thereby leading to uncomfortable fitting and facile the seepage of water when the swimming goggles are in use.

Referring to FIG. 2 and FIG. 3, the other two type of a pair of swimming goggles 6, 7 which have two straps that be respectively connected to a lens frames of each swimming goggles. As FIG. 2 the swimming goggles 7 have two lens frames 70,71 which are respectively connected together with interlacing by two straps 72. During adjusting, it is necessary to take off the swimming goggles 7 firstly, and then respectively adjusted the strap 72 adapt to the size of the nose bridge of wearer, finally wearing the swimming goggles 7 on the head of wearer. This way is inconvenient to wearer that should take off the swimming goggles 7 every adjusting, more particularly to the swimming goggles 7 is same situation with the aforementioned swimming goggles 8 which is adjusted one of the lens frame 70 (or the lens frame 71) to be in conformity with the periphery of right eyehole (or left eyehole) will move the lens frame 71 (or the lens frame 70) during the swimming goggles are worn on the wearer's eyeholes. In other words, both the lens frames 70,71 never respectively match wearer's eyeholes contour.

As show in FIG. 3, the swimming goggles 6 are connected a lens frames 60(61) by two straps 62 and the two straps 62 are clipped by a position element 63, thereby to provide the two straps 62 securely position with the lens frames 60(61). During adjusting, it is necessary to take off the swimming goggles 6, besides it is also necessary to take off the position element 63 and should be repeated several times to do the adjusting and wearing. Moreover, the swimming goggles 6 are same situation with the aforementioned swimming goggles 7,8, it should be to happen that is adjust one of the lens frame 60 (61; not show) to be in conformity with the periphery of left eyehole (or right eyehole) will move the other lens frame 61 (60) during the swimming goggles 6 are worn on the wearer's eyeholes. In other words, both the lens frames 60(61) never respectively match wearer's eyeholes contour.

### OBJECTS OF THE INVENTION

The object of the present invention is to provide a swimming goggles that can overcome the drawback of the aforementioned prior art.

More specifically, the main object of the present invention is to provide a pair of swimming goggles which respectively facilitate adjusting the periphery of each eyehole of wearer and which never influence the adjusting span of the left lens frame and the right lens frame of a swimming goggle each other when the swimming goggle in adjusting, to provide securely matching with the periphery of wearer's eyeholes respectively and get more comfortable and prevent effectively the seepage of water when the swimming goggles are in use.

To achieve the above objects, the present invention is featured in that a swimming goggles have a connection base which be mounted on the periphery of the lens frames of a swimming goggles, and have a strap mean for passing through the connection base of each of lens frames, thereby providing each of lens frames can move on the connection base respectively, so as to adjust each of lens frames respectively and do not influence each other when the swimming goggles are adjustment.

According to the above features of the present invention, the strap mean includes a first strap and a second strap which are respectively passing through the connection base of each of lens frames, and have a bunching element between the first strap and the second strap for joining with the center of the first strap and the second strap.

Based on the above features of the present invention, the bunching element has a through hole which provide the first strap and the second strap passing through and join together.

Based on the above features of the present invention, the bunching element, the first strap and the second strap can be formed integrally one unit.

According to another features of the present invention, a free end of the first strap and the second strap have a receiving element for pressure tightly the free end of the first strap and the second strap at the left lens frame and the right lens frame closely.

### BRIEF DESCRIPTION OF THE DRAWINGS

Other features and advantages of the present invention will become apparent in the following detailed description of the preferred embodiments with reference to the accompanying drawings of which:

FIG. 1 to FIG. 3 are a perspective view of swimming goggles of the prior art;

FIG. 4 is a perspective view of the disassembled parts of the swimming goggles of the present invention;

FIG. 5 is a perspective view of the part assembled of the swimming goggles in FIG. 4;

FIG. 6 is a perspective view of the swimming goggles in FIG. 4;

FIG. 7 is a top view of wearing on user's head and adjusting the span of lens frames of the swimming goggles in FIG. 4;

FIG. 8 is a front view of wearing on user's head embodiment of the swimming goggles of the present invention;

FIG. 9 is a perspective view of the second embodiment of the present invention;

FIG. 10 is a perspective view of the third embodiment of the present invention;

FIG. 11 and FIG. 12 are a perspective view of the swimming goggles have took off the receiving element of in FIG. 6 and FIG. 9.

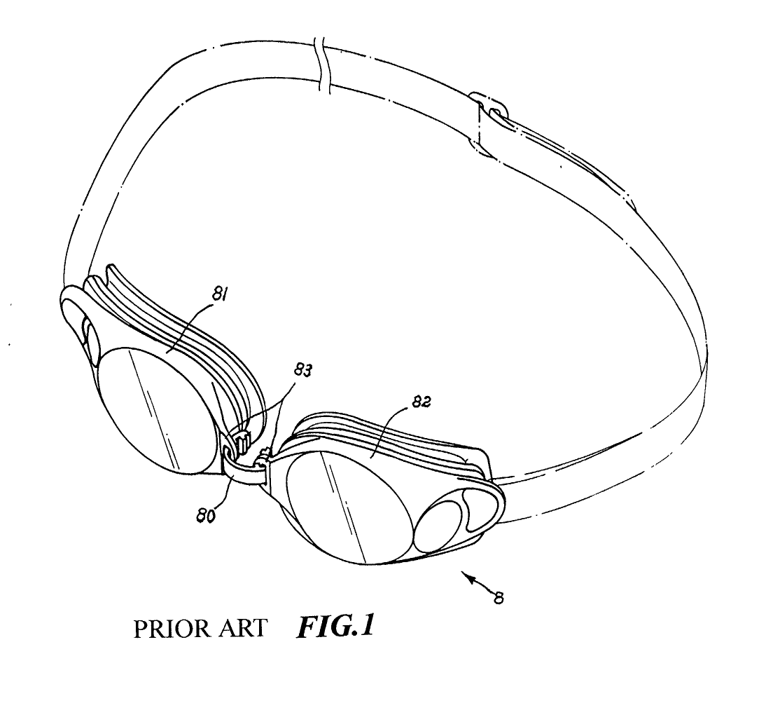

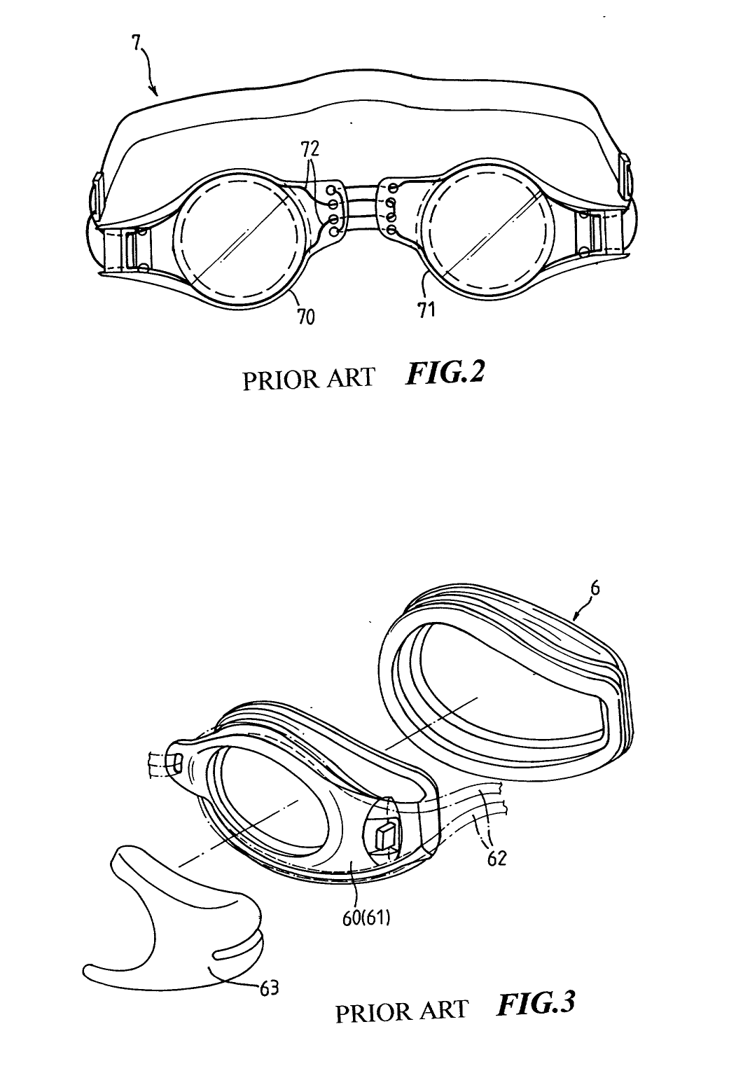

### DETAILED DESCRIPTION OF THE PREFERRED EMBODIMENT

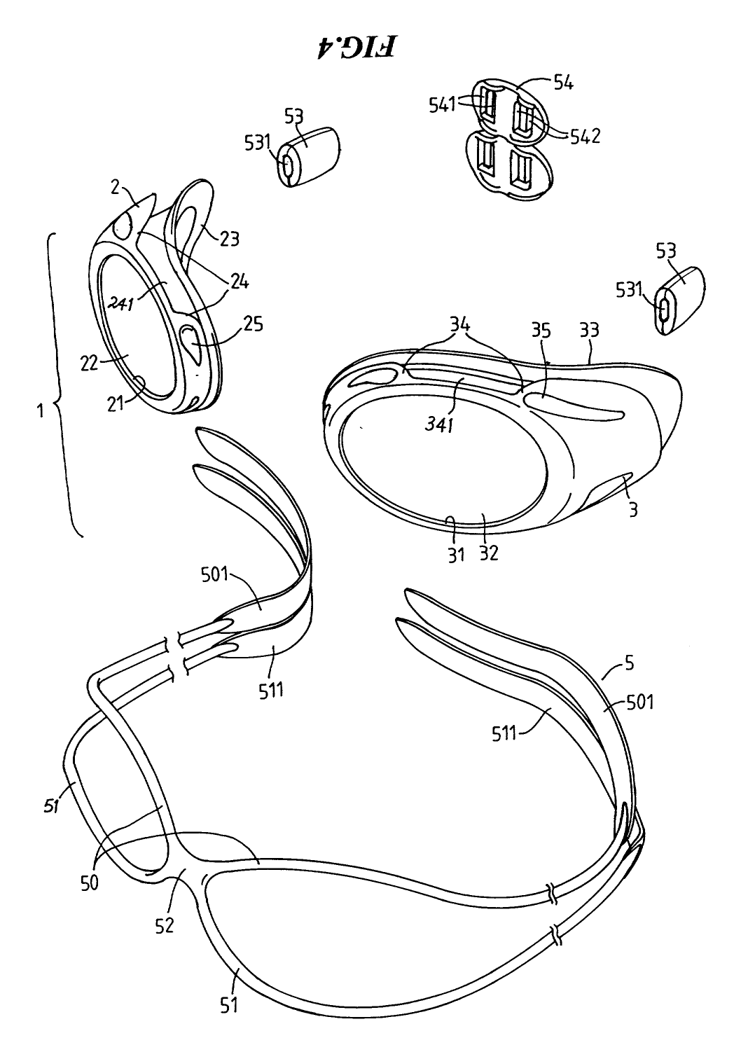

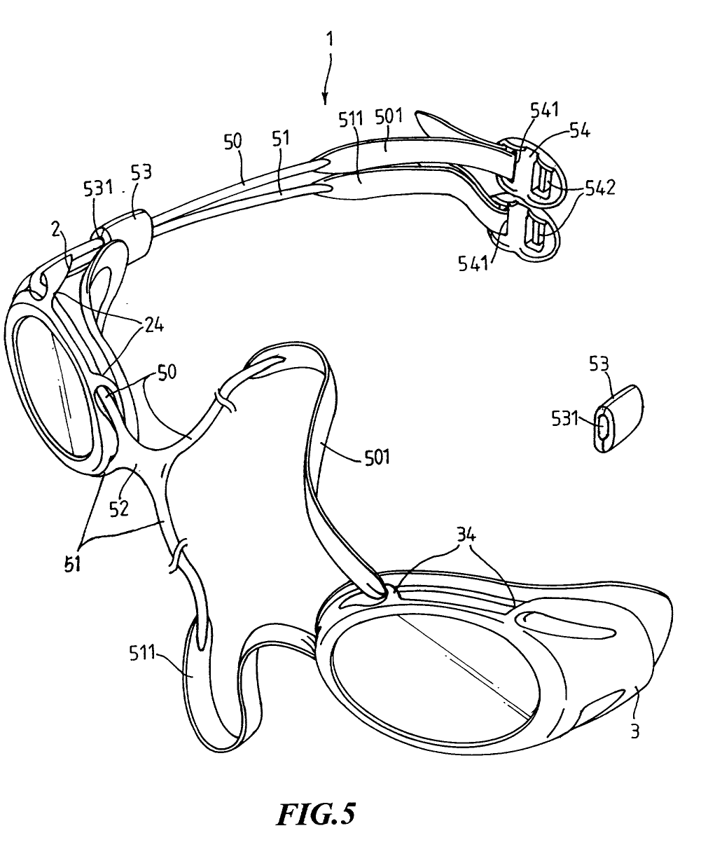

Referring to FIG. 4, the preferred embodiment of a swimming goggles 1 according to the present invention is show to comprise a right lens frame 2, a left lens frame 3 and a strap mean 5, wherein each of the lens frames 2,3 has a lens retaining spaces 21,31 for insert molding a lens units 22,32 thereon. The right lens frame 2 and the left lens frame 3 respectively has a gaskets 23,33 each of which is formed integrally with far away from the side of each of the lens units 22,32 for touching the periphery of a wearer's eyehole. The up and down periphery of the lens frames 2,3 have a connection bases 24,34 (only show in the up periphery of each lens frame) each of which is a slots 25,35 that is formed recess toward on the each lens frame 2,3 in this embodiment, as show in FIG. 4, the connection base 24,34 have a openings 241,341 each of which is respectively mounted on the connection bases 24,34 for facility a first strap 50, a second strap 51 of the strap mean 5 passing through the slots 25,35 respectively.

The strap mean 5 includes a first strap 50, a second strap 51 and a bunching element 52, wherein the first strap 50 and the second strap 51 are usually made of rubber and the bunching element 52 is formed integrally with the first strap 50 and the second strap 51 that are combined the first strap 50 and the second strap 51 at the middle in which the swimming goggles 1 is divided forming two areas by the bunching element 52. Moreover, the first strap 50 and the second strap 51 have a free ends 501,511 which are usually a plane shape and the each straps 50,51 is generally rotundity in cross section. The strap mean 5 further includes a receiving element 53, a fastener 54, wherein the receiving element 53 is a sleeve that have two through hole 531 thereon for combination the free ends 501,511 of each of the straps 50,51 together. The fastener 54 which has a adjusting hole 541,542 that are located both side of the fastener for passing through and adjusting the free ends 501,511 of each of the straps 50,51 respectively.

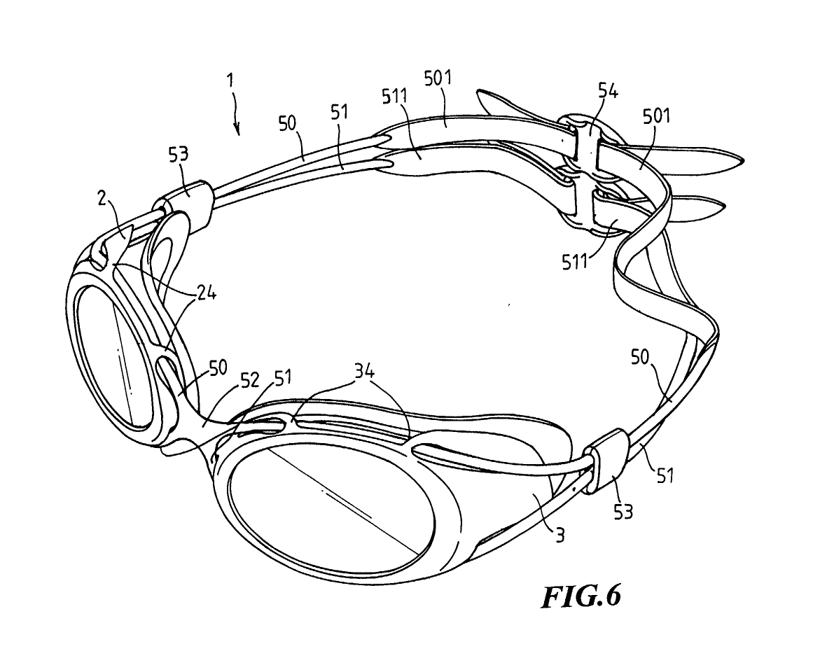

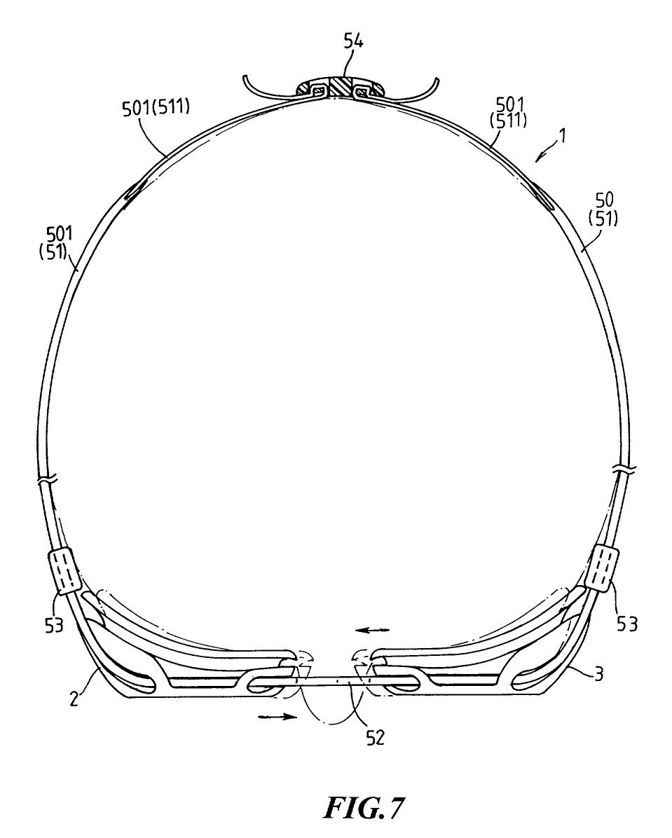

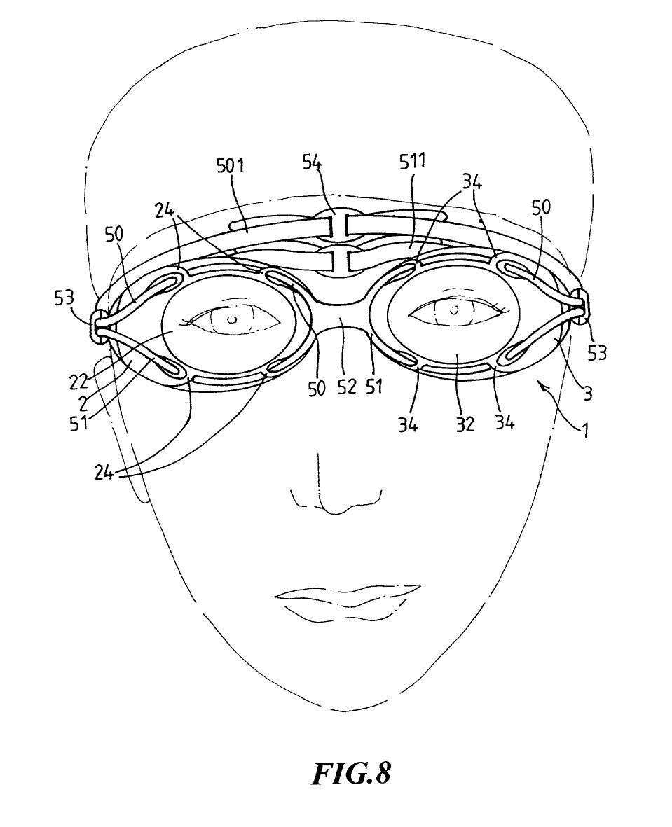

Referring to FIG. 5 and FIG. 6, during assembly, taking the free ends 501,511 of the first strap 50 and the second strap 51 are respectively passing through the connection bases 24,34 of the right lens frame 2 and the left lens frame 3. As show in FIG. 5, the free ends 501,511 of the first strap 50 and the second strap 51 are assembled which have passed through the connection base 24,the receiving element 53 and the adjusting hole 541 of the fastener 54. Referring one more to FIG. 5, the left lens frame 3 is disassemble with the other free end of the first strap 50 and the second strap 51, during assembly, the free ends 501,511 of the first strap 50 and the second strap 51 are respectively passing through the connection base 34 of the left lens frame 3 and the through hole 531 of the receiving element 53, leading to the first strap 50 and the second strap 51 connection to the receiving element 53 together, and then dividing the first strap 50 and the second strap 51 pass through the adjusting hole 542 of the fastener 54 respectively, such get the same pattern in which the first strap 50 and the second strap 51 passing through the adjusting hole 541 of the fastener 54, as show the right lens frame 2 of in FIG. 5. Referring to FIG. 6, the free ends 501,511 of the first strap 50 and the second strap 51 are respectively passing through the adjusting holes 541,542 of the fastener 54 such that the first strap 50 and the second strap 51 can be securely mounted on the right lens frame 2 and the left lens frame 3. Again referring to FIG. 7and FIG. 8 are a top view and front view of wearing on user's head to adjusting the span of the lens frames embodiment of the swimming goggles of the present invention. Since the first strap 50, the second strap 51 are passing through the connection bases 24,34 of the right lens frame 2 and the left lens frame 3, thereby the right lens frame 2 and the left lens frame 3 can be respectively voluntary moved the first strap 50, the second strap 51 thereon and can not any influence each other, as well as the right lens frame 2 and the left lens frame 3 are not limited in adjusting angle, so as to adjust the right lens frame 2 and the left lens frame 3 matching the periphery of a wearer's eyehole directly without take off the swimming goggles 1 and never influence the adjusting span of the right lens frame 2 and the left lens frame 3 each other when the swimming goggle in adjusting (as show in FIG. 8). Referring to FIG. 7, it is obvious that the right lens frame 2 is moved toward the right (a broken line position and as show in arrows), the left lens frame 3 is without moved toward direction the right. On the contrary, the left lens frame 3 can be moved toward direction the left by itself (a broken line position and as show in arrows) so as to adjust the right lens frame 2 and the left lens frame 3 respectively and can not influence each other, to provide securely matching with the periphery of wearer's eyehole respectively, such that it can get more comfortable and prevent effectively the seepage of water when the swimming goggles are in use.

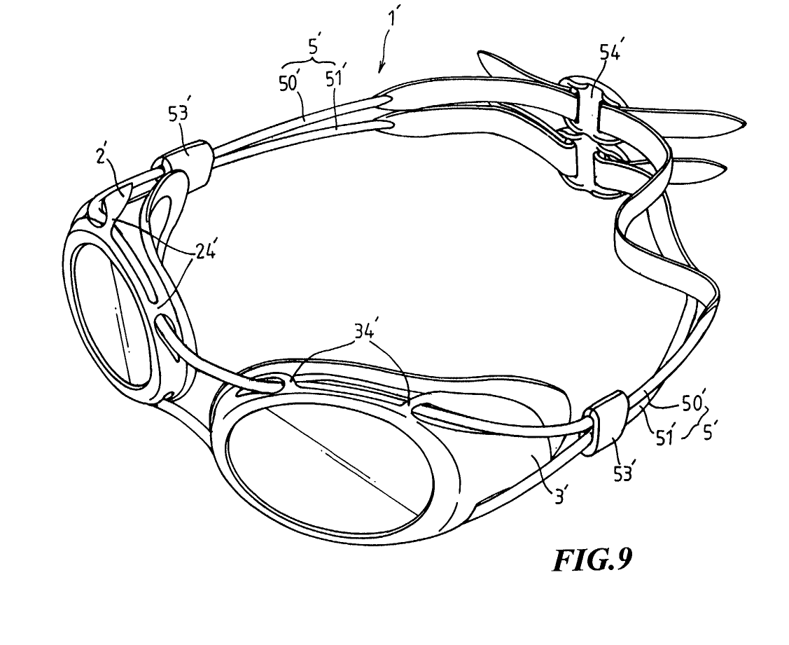

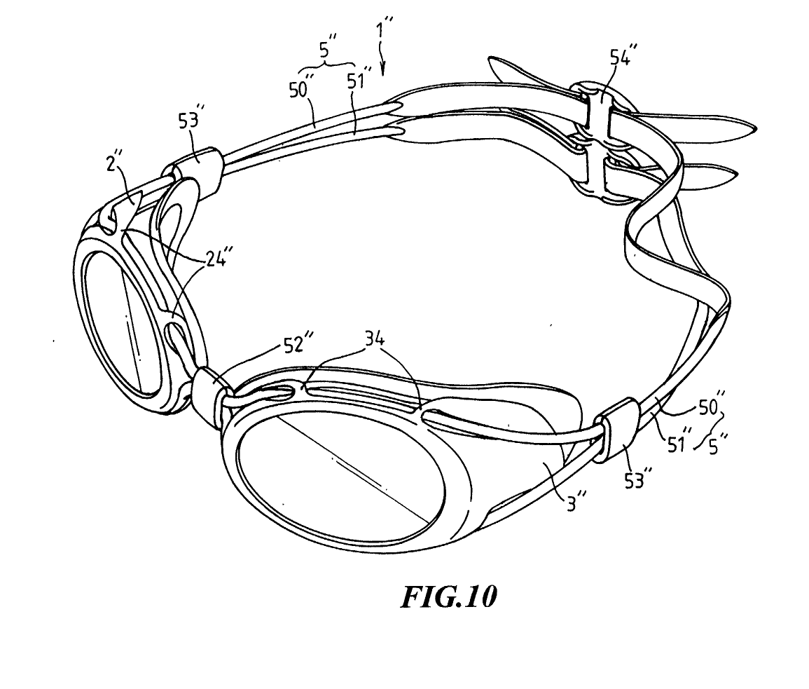

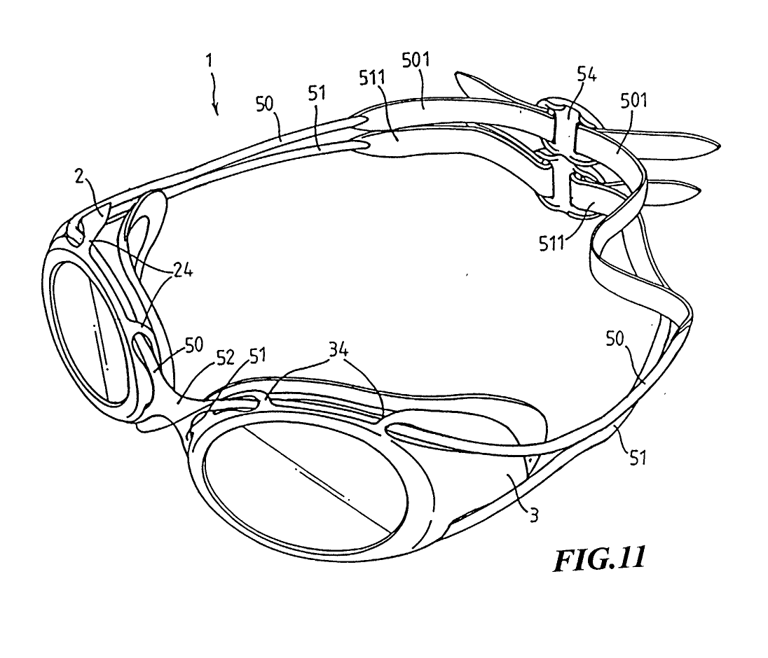

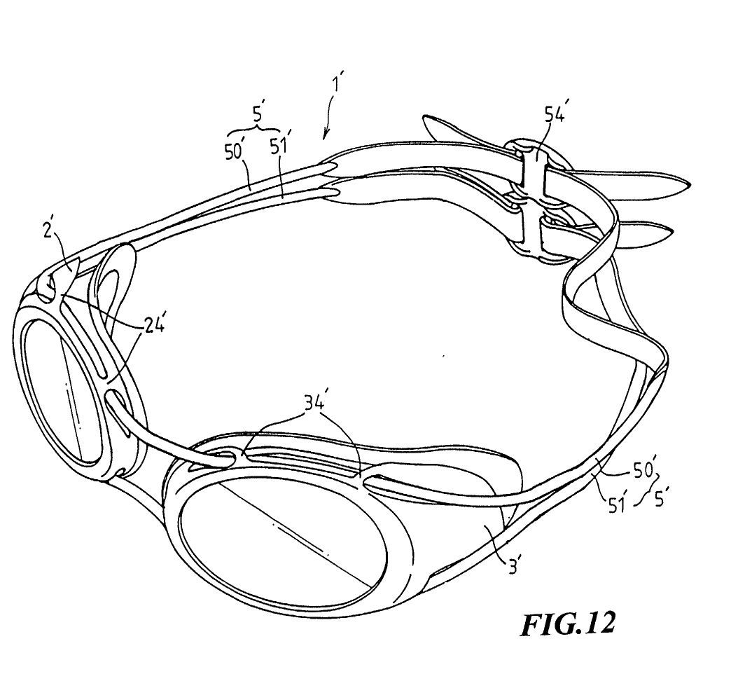

Referring to FIGS. 9-10 illustrate the second and third embodiment of a pair of swimming goggles 1′, 1″ according to the present invention. As show in FIG. 9, the second embodiment is generally similar to the first preferred embodiment, comprising a right lens frame 2′, a left lens frame 3′ and a strap mean 5′, the main different residing in the construction of the first strap 50′ and the second strap 51′ of the strap mean 5′ have not a bunching element, the rest parts e.g. a connection bases 24′,34′, of the right lens frame 2′, a left lens frame 3′, a receiving element 53′ which is a sleeve, a fastener 54′ are all same the first preferred embodiment, such that the right lens frame 2′ and the left lens frame 3′ can be respectively voluntary moved the first strap 50′ and the second strap 51′ thereon and can not any influence each other. In addition, As show in FIG. 10, the swimming goggles 1″ is the third embodiment which is generally similar to the second preferred embodiment, comprising a right lens frame 2″, a left lens frame 3″ and a strap mean 5″, the main different residing in the construction of the first strap 50″ and the second strap 51″ of the strap mean 5″ have a bunching element 52″ in center of each strap that the bunching element 52″ is same the sleeve 53 which has showed the first preferred embodiment for combination the first strap 50″ and the second strap 51″ together, the rest parts e.g. a connection bases 24″,34″, of the right lens frame 2″, a left lens frame 3″, a receiving element 53″ and a fastener 54″ are all same the first preferred embodiment, such that the right lens frame 2″ and the left lens frame 3″ can be respectively voluntary moved the first strap 50″ and the second strap 51″ thereon and can not any influence each other when the lens frames 2″,3″ are in adjustment. In addition to , the receiving elements 53,53′ of in FIG. 6 and FIG. 9 should be taking off from the first straps 50,50′ and the second straps 51,51′ and each lens frames 2,3 and 2′,3′ can be still respectively voluntary moved the first strap 50,50′ and the second strap 51,51′ thereon and can not any influence each other when the lens frames 2,3 and 2′,3′ are in adjustment.

While the present invention has been described in connection with what is considered the most practical and preferred embodiment, it is understood that this invention is not limited to the disclosed embodiments but is intended to cover various arrangements, which the strap mean comprise only one strap that is passing through the left lens frame and the right lens frame, or the connection base is protrude on the left lens frame and the right lens frame, or the connection base is mounted on the left side and right side of the periphery of each lens- frames etc., included within the spirit and scope of the broadest interpretation so as to encompass all such modifications and equivalent arrangements.

What is claimed is:

1. A pair of swimming goggles comprising:
a left lens frame and a right lens frame, each of which has an inner periphery that defines a lens retaining space for receiving a lens unit and the periphery of each lens frame have at least one connection base; and
a strap mean for passing through the connection base of each of lens frames and tightening the swimming goggles to a wearer's head.

2. The swimming goggles as claimed in claim 1, wherein the connection base is respectively mounted on the up and down periphery of the left lens frame and the right lens frame.

3. The swimming goggles as claimed in claim 2, wherein the strap mean include a first strap and a second strap which are passing through the up and down periphery of the left lens frame and the right lens frame.

4. The swimming goggles as claimed in claim 3, wherein a bunching element which is disposed between the first strap and the second strap for joining with the center of the first strap and the second strap

5. The swimming goggles as claimed in claim 4, wherein the bunching element, the first strap and the second strap are formed integrally.

6. The swimming goggles as claimed in claim 5, wherein the connection base is a slot which be formed recess toward the left lens frame and the right lens frame thereon in which the connection base have a opening that are respectively mounted on the up and down periphery of each of lens frames.

7. The swimming goggles as claimed in claim 4, wherein a free ends of the first strap and the second strap cooperatively has a receiving element which is a sleeve that have at least a through hole thereon for combination the first strap and the second strap at the left lens frame and the right lens frame closely.

8. The swimming goggles as claimed in claim 4, wherein the bunching element is a sleeve which have at least a through hole for combination the first strap and the second strap together.

9. The swimming goggles as claimed in claim 4, wherein the free ends of each of the first strap and the second strap are a generally plane shape, and between the free ends of each of the first strap and the second strap is generally rotundity in cross section.

10. The swimming goggles as claimed in claim 1, wherein a contact side of each of the lens frames has a gasket which is formed integrally with the contact side of each of the lens frames.

11. The swimming goggles as claimed in claim 1, wherein the strap mean further include a fastener which have a adjusting holes that are located both side of the fastener for passing through and adjusting the free end of the each strap respectively.
